(19) United States
(12) Patent Application Publication
(10) Pub. No.: US 2016/0082138 A1
(43) Pub. Date: Mar. 24, 2016

(54) ULTRAVIOLET DISINFECTION UNIT

(71) Applicants: James R. KERMODE, Los Altos, CA (US); Justin A. LANCE, Hollister, CA (US); Julia A. RASOOLY, San Francisco, CA (US); Michael RASOOLY, San Francisco, CA (US); Charles Martin SCHWIMMER, San Jose, CA (US)

(72) Inventors: James R. KERMODE, Los Altos, CA (US); Justin A. LANCE, Hollister, CA (US); Julia A. RASOOLY, San Francisco, CA (US); Michael RASOOLY, San Francisco, CA (US); Charles Martin SCHWIMMER, San Jose, CA (US)

(21) Appl. No.: 14/857,522

(22) Filed: Sep. 17, 2015

Related U.S. Application Data
(60) Provisional application No. 62/052,164, filed on Sep. 18, 2014.

(51) Int. Cl. A61L 2/10; A61M 39/18

(52) U.S. Cl. CPC A61L 2/10; A61M 39/18; A61M 2039/167

(57) ABSTRACT

An ultraviolet (UV) disinfection unit for disinfecting catheter line connections is provided. The unit can be used to disinfect solution set and transfer set catheters used for peritoneal dialysis. The unit can comprise a concavely curved bottom surface. A tubing trough of the unit can allow for ambidextrous positioning.

### CROSS REFERENCE TO RELATED APPLICATION

This application claims the benefit under 35 U.S.C. §119 of U.S. Patent Application No. 62/052,164 filed Sep. 18, 2014, titled “ULTRAVIOLET DISINFECTION UNIT”. This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates generally to sterilization units, more particularly sterilization of connectors used in a medical application, for example during peritoneal dialysis (PD).

### BACKGROUND

Peritoneal dialysis can be used as a treatment for patients with severe chronic kidney disease. Fluid is introduced through a tube in the abdomen and flushed out periodically either while the patients sleeps, in automated peritoneal dialysis or during regular dialysis sessions through the day, as in continuous ambulatory peritoneal dialysis.

As shown in FIG. 1, a patient undergoing peritoneal dialysis can have an indwelling catheter surgically inserted into the abdomen. A transfer catheter 2 can be attached to the indwelling catheter 4. The transfer catheter can be replaced, in a sterile environment, such as at a clinic, every few months to a year. Between dialysis sessions, the patient wears the transfer catheter against the body. The overall size and shape of the catheter greatly influences patient comfort between dialysis sessions. Smaller catheters have the further benefit of being less conspicuous underneath the patient's clothes. During dialysis sessions, the transfer catheter can be connected to a drain bag to drain fluid present in the abdomen and a fresh dialysate bag to introduce fluid to the abdomen. The drain bag and dialysate bags can be attached sequentially or can be attached in parallel, using a Y-shaped solution set catheter 6. Prior to each treatment, the patient connects the tip of the transfer catheter to new dialysis tubing using rigorous aseptic techniques to maintain sterility. The same sterile technique must be employed when disconnecting the catheters as well. Once the patient feels confident enough to perform the procedure at home, and after many months of practice, the time to connect and start PD takes 10-30 minutes. The patient and anyone around them must always wear a mask, close doors and windows, turn off fans, and thoroughly wash hands for 2 minutes prior to connecting or disconnecting catheters. If sterility is compromised at any time, the component being used must be replaced and the whole process started again.

This is obviously a complicated and time-consuming process that is highly reliant on patient compliance. If a patient fails to adhere to any of the strict steps of the sterilization procedure, he or she faces a greatly increased risk of a serious infection, commonly referred to as peritonitis. This type of internal infection, if not caught early, may lead to sepsis and death of the patient. Typically, peritoneal dialysis (PD) patients experience a 50% chance of infection during the first 12 to 18 months and experience 15% mortality/yr directly related to the infection. In addition to seriously endangering a patient's health, infections in peritoneal dialysis are also very costly to treat. The average total charges from a peritonitis hospital stay are roughly $50,000 dollars and the entire annual cost on the healthcare system is around $1.5 billion. Given that the noncompliance rate for a standard peritoneal dialysis procedure is around 30%, there is a huge need to help reduce the health and financial burdens of infection.

Ultraviolet (UV) disinfection systems are known in the art. U.S. Pat. Nos. 4,882,496; 7,834,328; 4,620,845; 6,461,568 and U.S. Publication Nos. 2005/0013729 and 2007/0274879, the disclosures of which are incorporated by reference herein in their entireties, describe such systems. However, such systems can be cumbersome, making them difficult for a user to use. Additionally, such systems tend to rely on UV disinfection for complete disinfection, which can, in the absence of proper components and connectors, limit the effectiveness of the disinfection.

### SUMMARY OF THE DISCLOSURE

In some embodiments, a UV disinfection unit for a catheter line connection is provided. The unit comprises a housing comprising a concavely curved bottom surface; a lid shaped to mate with a top open part of the housing, the lid connected to the housing by a hinge; a UV source positioned within the unit; a tubing trough positioned beneath the lid and configured to receive a catheter connector connecting two catheters; and an exposure chamber positioned along the tubing trough, the exposure chamber at least partially exposed to the UV source.

In some embodiments, the disinfection unit comprises a first recess positioned along the tubing trough on a first side of the exposure chamber and a second recess positioned along the tubing trough on a second side of the exposure chamber, the first and second recesses shaped to receive a protrusion on the catheter line connection and stabilize the catheter line connection within the tubing trough. The UV source can comprise 4 lamps. The unit can comprise a lid closed sensor. The hinge can comprise a magnetic hinge. The exposure chamber can comprise a volume of about 15 cc. The disinfection unit can comprise a volume of about 5.5 in³. The lid can comprise a graphic overlay configured to be backlit by the UV source. The lid can be configured to block access to a power control when the lid is in a closed position.

In some embodiments, a UV disinfection unit for a catheter line connection is provided. The disinfection unit comprises a housing; a lid shaped to mate with a top open part of the housing, the lid connected to the housing by a hinge; a UV source positioned within the unit; a tubing trough positioned beneath the lid and configured to receive a catheter line connection; an exposure chamber positioned along the tubing trough, the exposure chamber at least partially exposed to the UV source; a protrusion along the catheter line connection; a recess positioned along the tubing trough within the exposure chamber, the recess shaped to receive the protrusion and stabilize the catheter line connection within the tubing trough.

In some embodiments, the disinfection unit comprises a first recess positioned along the tubing trough on a first side of the exposure chamber and a second recess positioned along the tubing trough on a second side of the exposure chamber, the first and second recesses shaped to receive the protrusion and stabilize the catheter line connection within the tubing trough. The disinfection unit can comprise a concavely curved bottom surface. The exposure chamber can be configured to receive a transfer valve comprising external UV-transparent channels.

In some embodiments, a UV disinfection unit for a catheter line connection is provided. The disinfection unit comprises a housing; a lid shaped to mate with a top open part of the housing, the lid connected to the housing by a hinge; a UV source positioned within the unit; a tubing trough positioned beneath the lid and configured to receive a catheter line connection; an exposure chamber positioned along the tubing trough, the exposure chamber at least partially exposed to the UV source; a protrusion along the catheter line connection; a first recess positioned along the tubing trough on a first side of the exposure chamber, a second recess positioned along the tubing trough on a second side of the exposure chamber, the first and second recesses shaped to receive the protrusion and stabilize the catheter line connection within the tubing trough.

The housing can comprise a concavely curved bottom surface. The UV source can comprise 4 lamps. The unit can comprise a lid close sensor. The hinge can comprise a magnetic hinge. The exposure chamber can comprise a volume of about 15 cc. The disinfection unit can comprise a volume of about 5.5 in3. The lid can comprise a graphic overlay configured to be backlit by the UV source. The lid can be configured to block access to a power control when the lid is in a closed position.

In some embodiments, a method for disinfecting a catheter line connection is provided. The method comprises placing a UV disinfection unit on a thigh of the user such that the concavely curved bottom surface of the unit is positioned on the user's thigh; opening a lid of the disinfection unit; positioning a catheter line connection within a tubing trough of the unit such that the portion to be disinfected is placed within an exposure chamber comprising a portion of the tubing trough deeper than other portions; and exposing the exposure chamber to UV light.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates a conventional peritoneal dialysis setup.

FIG. 2A illustrates a front view of an embodiment of a disinfection unit

FIG. 2B illustrates a top view of an embodiment of a disinfection unit.

FIG. 2C shows a finger recess used to open the lid of an embodiment of a disinfection unit.

FIG. 3A illustrates a right side view of an embodiment of a disinfection unit, showing the tubing trough (Light block element is not shown for clarity).

FIG. 3B illustrates a top view of an embodiment of a disinfection unit, again showing the tubing trough.

FIG. 3C illustrates a cross-sectional view, through the tubing trough of an embodiment of a disinfection unit.

FIG. 3D illustrates an exposure chamber element.

FIG. 3E illustrates a top right view of an embodiment of a disinfection unit and highlights a light blocking element within the tubing trough.

FIGS. 3F and 3G illustrate an embodiment of a light block.

FIG. 4A illustrates a cross-sectional view of an embodiment of a disinfection unit.

FIG. 4B illustrates an embodiment of a magnetic hinge as used on a disinfection unit.

FIG. 4C illustrates a cross-sectional view of an embodiment of a disinfection unit and shows a magnetic lid latch mechanism.

FIG. 5 illustrates an embodiment of a lid closed sensor.

FIGS. 6A and 6B illustrate an embodiment of a lid and the relationship between the lid opening and a control panel for use with a disinfection unit.

FIG. 6C illustrates an embodiment of a control panel.

FIG. 7A illustrates the relationship of various internal components of an embodiment of a disinfection unit.

FIG. 7B illustrates an embodiment of a disinfection unit highlighting the relative position of some of the batteries.

FIG. 7C illustrates a cross sectional view of an embodiment of a disinfection unit highlighting a flexible rib, internal to the unit, which retains one of the inverters.

FIG. 8A illustrates a cross-sectional view of an embodiment of a disinfection unit showing the relative location of the UV lamps.

FIG. 8B illustrates the relative orientation of the UV lamps in an embodiment of a disinfection device.

FIG. 8C illustrates a cross-sectional view of an embodiment of a disinfection device, highlighting the exposure chamber.

FIG. 9A illustrates a cross-sectional view of an embodiment of a disinfection unit, highlighting the overmolding on the battery door and its relationship with the bottom housing.

FIG. 9B illustrates a battery door of an embodiment of a disinfection device, highlighting the contour of the underlying rigid plastic component.

FIG. 9C illustrates a right side view of an embodiment of a disinfection device, highlighting the relative heights of the battery door overmold and the bottom of the housing.

FIG. 9D illustrates a detail view of a bottom housing of an embodiment of a disinfection device.

FIG. 9E illustrates a detail view of a battery door of an embodiment of a disinfection device.

FIG. 9F illustrates a bottom view of an embodiment of a disinfection device highlighting an opening to allow access to the underside of a printed circuit board.

FIG. 10 illustrates another embodiment of a disinfection device showing the relationship between a disinfection device and a catheter during disinfection.

FIG. 11 illustrates an embodiment of a backlit graphic overlay.

FIG. 12 illustrates a perspective view of a solution set shoulder.

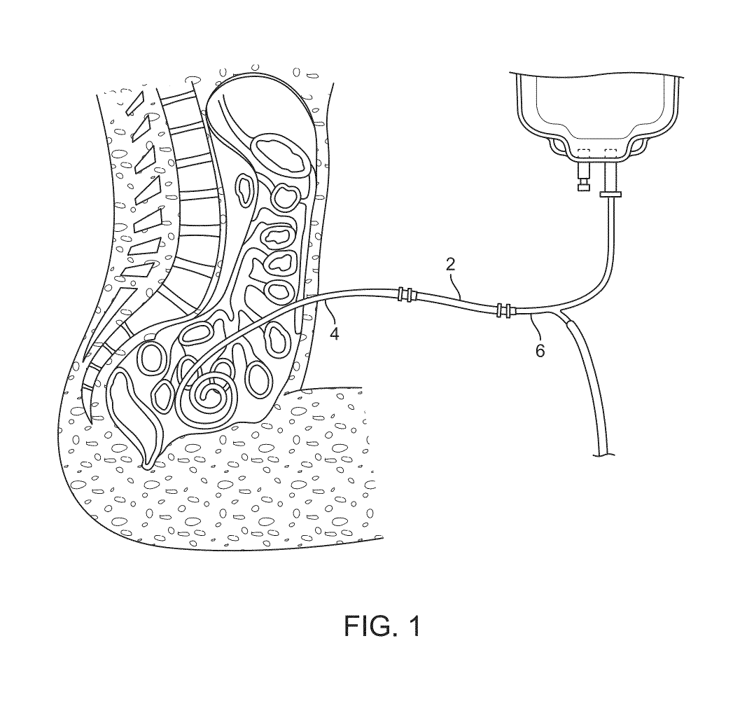

### DETAILED DESCRIPTION

Embodiments of the ultraviolet (UV) disinfection system disclosed herein can be used by peritoneal dialysis (PD) patients. The connection between the transfer catheter 2 and the solution set catheter 6 can be placed in the UV disinfection unit and sterilized through exposure to UV light. While peritoneal dialysis is described in particular, it will be appreciated that the systems, methods, and devices disclosed herein can be used in other applications, for example other applications using catheter line connections, such as Central Venous Catheters (CVC) and Peripherally Inserted Central Catheters (PICC).

The disinfection unit described herein can provide a small volume unit that adds a further level of convenience for the user during sterilization. Traditionally, UV sterilization units were large and bulky (e.g., about 12″×6″×6″). The UV disinfection unit described herein can have a small footprint (e.g., about 6″×3″×3″, with a volume of about 5.5 cubic inches, and weigh about ¼ pound), allowing portability and lap-top use. The disinfection unit can have a small footprint by minimizing the disinfection area or exposure chamber within the unit. The disinfection unit can have features (e.g., grooves or recesses in the tubing trough) to help ensure the portion of the catheter connection to be disinfected is properly situated within the exposure chamber. Additionally, the disinfection unit can be used in conjunction with catheter line connections comprising valves or other components comprising a small volume to be disinfected. This small disinfection volume can lead to a reduction in all associated volumes; smaller lamps, smaller lamp power supplies, smaller batteries and thereby smaller enclosure.

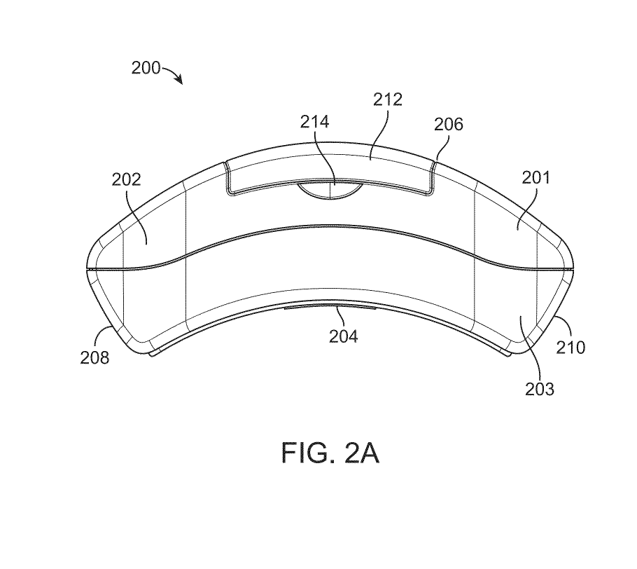

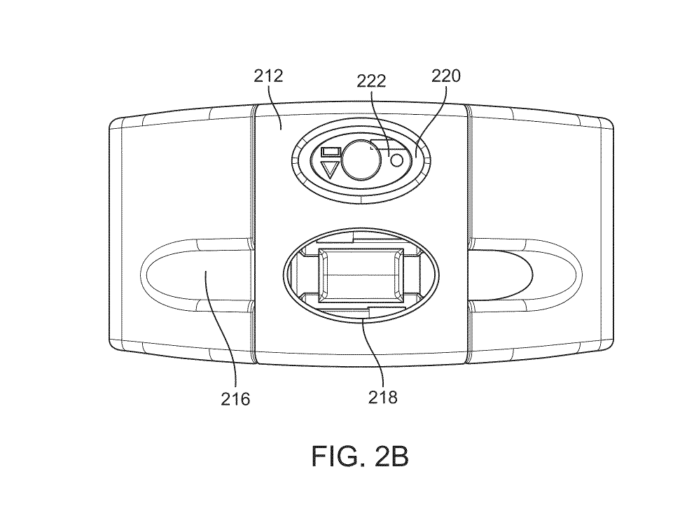

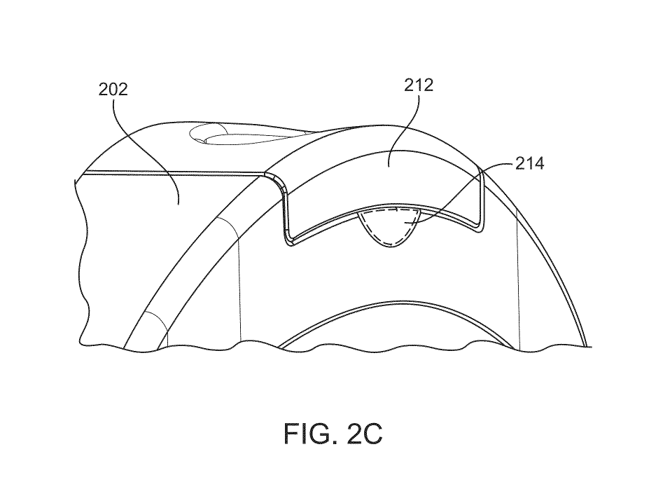

FIG. 2A illustrates a front view of an embodiment of a disinfection unit 200. The disinfection unit comprises a housing 202 and a lid 212. The housing can comprise an upper housing 201 and a lower housing 203. The upper housing may be coupled to the lower housing (e.g., by one or more hinges or joints) or, optionally, the upper housing may be removably attached to the lower housing. Being able to separate the upper housing 201 and lower housing 203 can allow access to and maintenance of internal components of the unit 200.

The bottom housing 203 is shown as having a concavely curved bottom surface 204. In one embodiment, the contour of the curved bottom surface 204 is adapted and configured to allow the unit 200 to conform to the leg of a user. Allowing the unit 200 to be placed on the upper thigh or user's lap during disinfection can provide convenience to a user during and before dialysis sessions. As described above, the current sterilization process is burdensome and requires preparation of the surrounding environment. The UV disinfection unit described herein negates the need for such rigorous procedures, allowing dialysis to be performed in various situations and environments. The ability to place the disinfection unit 200 on the lap enhances user convenience. For example, a user does not need a particular table top surface to perform dialysis with a lap-top unit. Additionally, the user does not need to lean over to a table or stand to perform sterilization prior to dialysis.

Existing UV disinfection units are configured to be placed on a table, and can have a flat bottom surface. Such units can be hard to place on the lap as the smooth and/or flat lower surface can cause the unit to slip off the lap, leading to insecure lap-top positioning of the unit 200.

As shown in FIG. 2A, the top surface 206 can also be curved. The top and bottom surfaces being curved can allow for more compact design of internal components of the unit 200. A compact design can allow for a smaller unit that can be easier to carry and use. Other configurations are also possible. For example, the top surface may not be curved.

The corners and edges between the top surface 206 and bottom surface 204 and the end surfaces 208, 210 can be rounded which can help reduce the size of the unit 200. The end surfaces 208, 210 can be generally perpendicular to the curve of the top surface 206 and bottom surface 208. This configuration can allow for an overall reduced volume. Other configurations are also possible. The unit 200 can have square corners, in some embodiments.

The housing 202 comprises a lid 212 on the top surface 206 of the unit 200. The lid 212 can be opened to allow placement of the catheter connection within the unit 200. The housing 202 can comprise a recess or groove 214 which can create a finger hold to facilitate easy opening of the lid 212. The recess 214 is shown in greater detail in FIG. 2C

FIG. 2B illustrates a top view of the disinfection unit 200. FIG. 2B shows the lid 212 positioned over the tubing trough 216. The lid can include a window 218 to allow visualization of the catheter connection positioned in the tubing trough 216. The window can comprise PolyMethylPentene (PMP) or Cyclic Olefin Copolymer (COC). These materials can degrade with long term exposure to UV light. Thus, their use can depend on the anticipated life-cycle of the disinfection unit. Other materials, such as quartz, can also be used. Quartz can exhibit higher endurance upon repeated exposure to UV light than the other described materials. In some embodiments, the unit 200 does not include a window on the lid. The lid 212 can also comprise an opening 220 to allow access to a control panel 222, described in greater detail below, positioned on the unit 200. The opening 220 can comprise a beveled edge which can help ensure UV light from the exposure chamber is blocked or scattered prior to reaching the control panel.

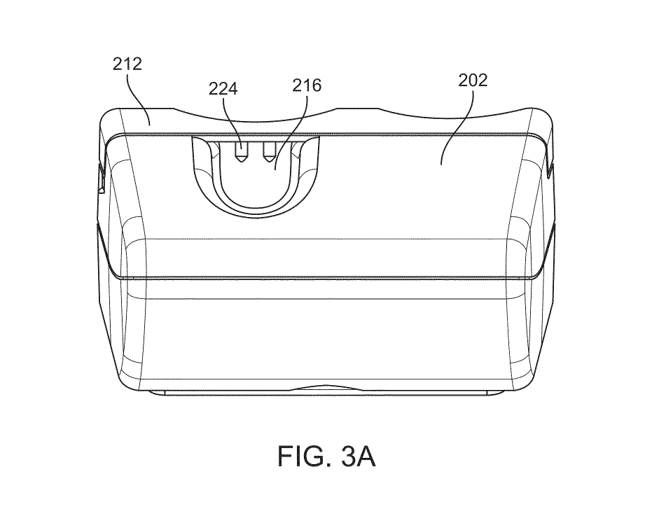

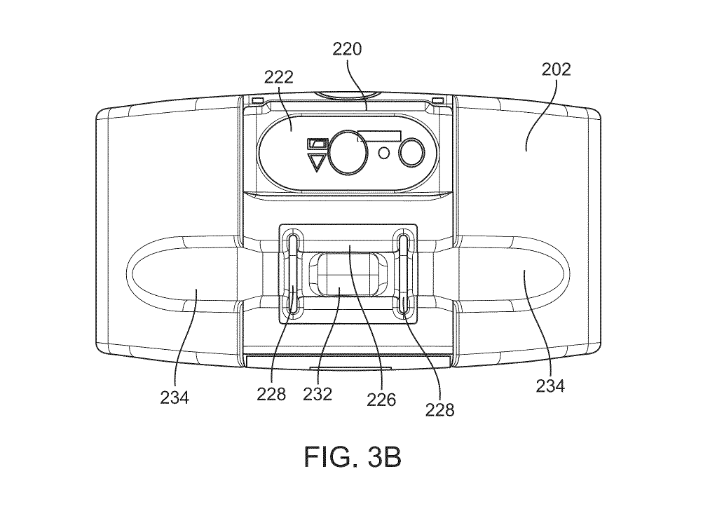

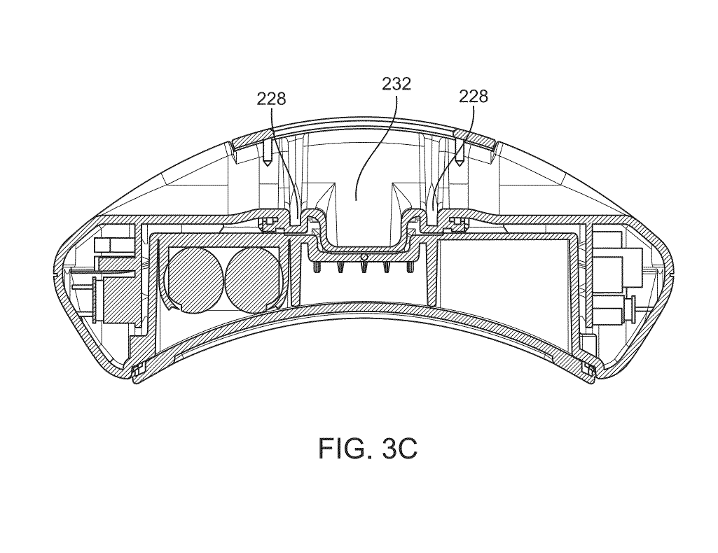

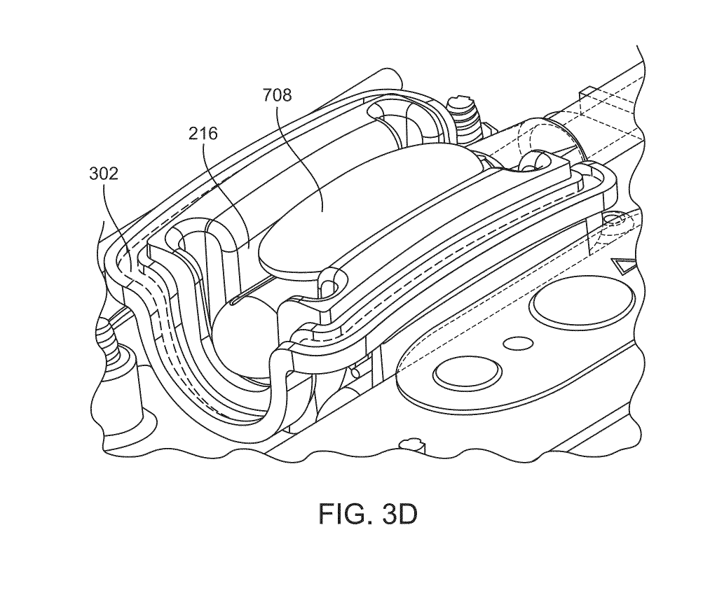

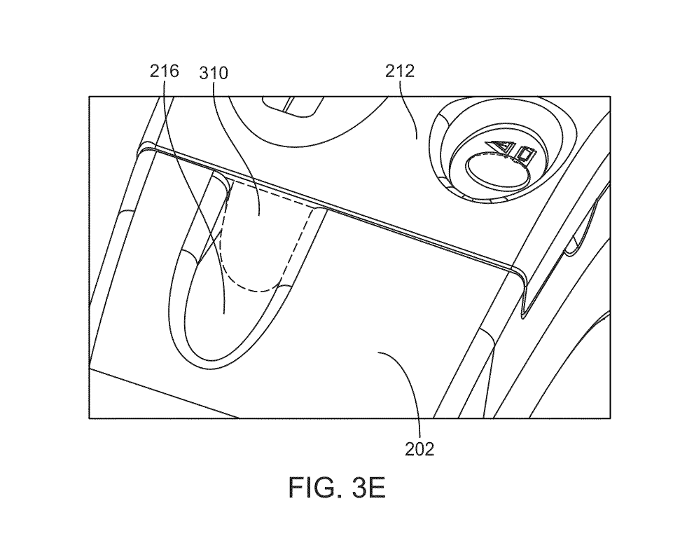

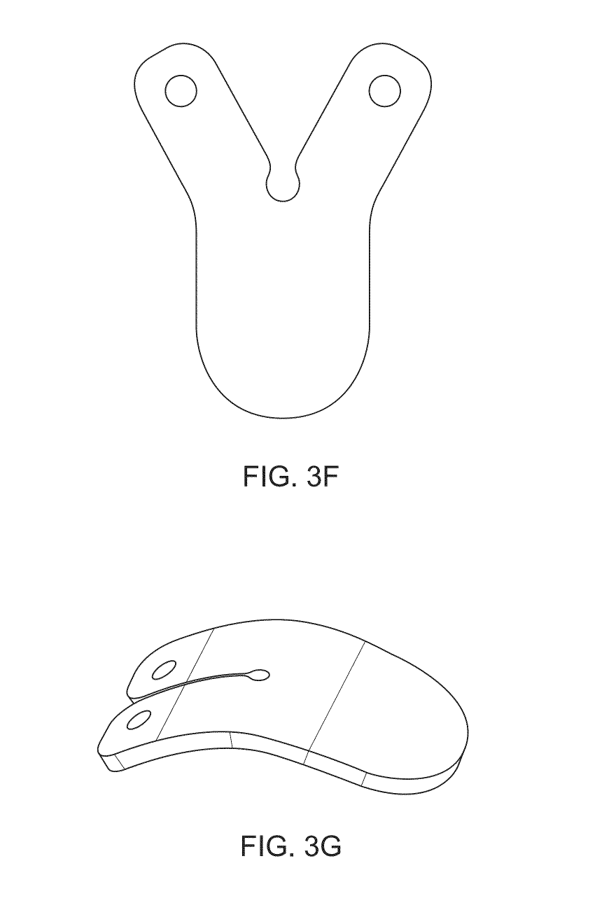

FIGS. 3A and 3B illustrate an embodiment of the tubing trough 216 in more detail. FIG. 3A shows an end view of the unit 200, including the tubing trough 216. FIG. 3A shows the lid 212 and the housing 202. The lid 212 comprises two prongs 224 extending down into the tubing trough. The prongs 224 are used to orient and help secure the light blocks 310 (not shown for clarity). The tubing trough can comprise a rounded bottom surface, as shown in FIG. 3A, for example, to conform to the round shape of a catheter. Other configurations are also possible. For example, the tubing trough 216 can comprise a rectangular bottom surface.

FIG. 3B illustrates a top view of the housing 202 with the lid 212 removed. The control panel 222 on the housing 202 is shown. As shown in FIG. 3B, beneath the lid 212 is a central portion 226 of the tubing trough 216. The central portion 226 can be configured to receive a catheter connection. For example, a catheter connection comprising a small volume valve is described in U.S. Provisional Patent Application No. 62/008,433, filed Jun. 5, 2014, entitled TRANSFER CATHETER FOR ULTRAVIOLET DISINFECTION (“'433 Application”), the disclosure of which is herein incorporated by reference in its entirety. Features of the catheter systems and valves disclosed therein allow the catheter connections to comprise a small volume (e.g., 0.1-0.5 cc) to be disinfected, allowing a lower amount of disinfection light.

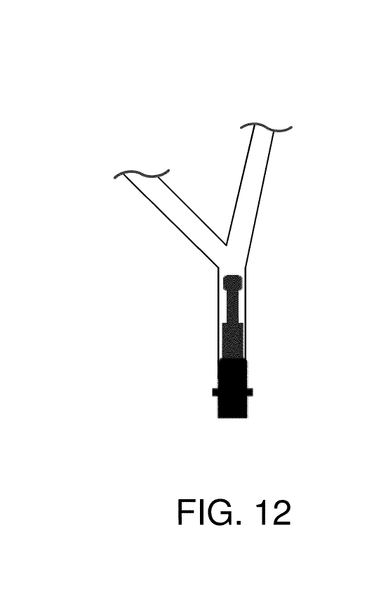

Other catheter connections are described in U.S. Provisional Patent Application No. 61/978,556, filed Apr. 11, 2014, entitled CONNECTOR DISINFECTION SYSTEM (“'556 Application”), the disclosure of which is herein incorporated in their entireties. This application also discloses small volume disinfection units and catheter systems. The central portion 226 can include grooves, recesses, and similar features to receive particular catheter connectors. For example, shown in FIG. 3B, are grooves 228, which can be shaped to receive a shoulder of a solution set catheter manufactured by Baxter International, Inc as used in their DIANEAL PD Solution ULTRABAG System (See FIG. 12). In some embodiments, the central portion 226 includes grooves or recesses to receive other catheter components, for example, the valves disclosed in the '433 and '556 Applications. The recesses can be configured to receive features of catheters or valves on the solution set side of the catheter connection or the transfer catheter side of the catheter connection.

The UV disinfection unit can also be used with other available dialysate catheter sets, for example, catheters comprising luer lock y-sets. For example, catheter sets manufactured by companies including J. Mitra & Co Pvt. Ltd., Claris Life Sciences, Baxter International, Inc., and Samen Pharmaceuticals are also contemplated.

In some embodiments, as shown in FIG. 3B, the central portion 226 comprises recesses or grooves 228 on both sides of the central portion 226. Positioning the grooves 228 on either side of the central portion 226 in this manner can allow for the catheter connection to be put into the unit 200 with the patient side catheter on the right side and the solution set catheter on the left side, or vice versa. This ambidextrous positioning of the catheter connection can allow a user comfort and convenience during disinfection and dialysis regardless of whether the user is right-handed or left-handed. The ambidextrous positioning can also allow user flexibility when determining a position for the dialysate and drain bags. For example, a user can leave the dialysate bag, which can be heavy, in place and choose their disinfection position based upon the position of the dialysate bag.

A catheter component such as a valve can be connected to the solution set side of the catheter connection. The valve can be connected to a valve or clamp on the transfer catheter side of the catheter connection. The grooves or recesses in the central portion 226 can be configured to receive a feature from a catheter or catheter component (e.g., a valve or clamp) from the solution set side of the catheter connection or the patient side of the catheter connection. As the solution set and transfer catheters will be connected during disinfection, a groove or recess to receive either side can help keep the entire catheter connection in place within the unit 200. The grooves or recesses can also help to ensure that the portion of the catheter connection to be disinfected (e.g., the valve) is properly in place within the exposure chamber of the unit 200. In this way, the grooves or recesses can help ensure the user properly positions the catheter connection within the unit. Proper positioning of the catheter connection within the unit can help ensure that the portion to be disinfected is properly positioned within the exposure chamber. Furthermore, the ambidextrous receiving feature can allow proper positioning of the catheter connection regardless of which way the catheter connection is inserted into the unit. Whether the connection is inserted in a right-handed or left-handed manner, the portion to be disinfected will be positioned within the exposure chamber and/or in substantially the same position.

In some embodiments, the central portion 226 can include multiple grooves, recesses, or features to receive particular catheter connectors. In some embodiments, the unit 200 comprises a modular central portion 226. The unit 200 can be compatible with a variety of removable central portions 226. The particular central portion 226 selected can depend on the type of catheter connector used. For example, in some embodiments, the central portion 226 can be configured to receive the Baxter solution set catheter and the valve described in the '433 application, incorporated by reference herein. In some embodiments, the central portion 226 can be configured to receive the Baxter solution set catheter and a standard transfer set valve or clamp. For another example, in some embodiments, the central portion 226 can be configured to receive a solution set catheter and the valve described in the '556 application. The unit can comprise slots or tracks configured to receive a removable central portion 226.

As shown in FIG. 3B, the central portion 226 can include a deeper portion 232, for example, to receive a larger portion of the catheter connection. This portion of the trough 216 can serve as an exposure chamber 232 as this portion extends down into the unit, allowing more exposure to UV light. The deeper sides of the exposure chamber can allow for placement of a larger feature, such as a valve (e.g., the valve described in the '433 Application). The exposure chamber 232 can also allow for precise positioning of particular valve components to allow maximum exposure to UV light, as the UV lights are positioned in close proximity to the exposure chamber 232 and configured to irradiate the exposure chamber. A small volume exposure chamber, defining the kill zone, as shown in FIG. 3B, can help to minimize the amount of UV light necessary to disinfect the catheter connection, which can help reduce lamp and power requirements and the footprint of the unit 200. In some embodiments, the volume of the exposure chamber is about 15 cc.

On either side of the exposure chamber recess 232 are the grooves 228 described above. Outside of the grooves 228 are shallower portions 234 of the trough, in which the catheter tubing on either side of the connection can be positioned. The shallower portions 234 of the trough 216 can be wider than the narrower central portion. The extra width can help create a tortuous path for UV light to travel around the light blocks, shown and described below.

A cross-sectional view of the unit 200, showing the exposure chamber recess 232, is also shown in FIG. 3C. The grooves 228 are positioned on either side of the exposure chamber recess 232.

FIG. 3D illustrates the tubing trough 216 with a transfer valve described in the '433 application positioned within the tubing trough 216. The transfer valve can comprise external UV-transparent channels to allow for UV disinfection. The exposure chamber comprises a groove 302 positioned around the tubing trough 216, in between the inner surface of the tubing trough 216 and the bottom surface of top housing 201 (Top housing 201 not shown for clarity). The gap or groove 302 between these surfaces allows for placement of silicone sealant which can ensure a water-tight seal between the exposure chamber 226 and the top housing 201.

In some embodiments, all or portions of the tubing trough 216 can include a rough surface finish, which can help scatter or disperse UV light to reduce direct UV-C exposure.

The unit 200 can comprise light blocks 310 configured to block the tubing trough 216, as shown in FIG. 3E. The light blocks 310 can be configured to block the trough 216 when no catheter is positioned within the trough 216. The block 310 can be configured to move out of the way when a catheter is in position within the trough 216. The light block 310 can comprise a flap of flexible material. The material can be configured to be UV opaque. In some embodiments the material can be configured to transmit visible light. In some embodiments, the light block comprises silicone rubber. The light block can comprise a shape configured to match the cut of the tubing trough. The light block may be made from a flat sheet of material formed in a generally Y-shape, as shown in FIG. 3F. The base of the Y may have a shape similar to the cross-section of the trough 216. The Light block may assume a curve when the arms of the Y are brought together and retained in that shape when the light block is secured to lid 212 with adhesive and pins 224, as shown in FIG. 3G. The curve of the light block may allow it to extend down into the tubing trough 216. The light block 310 can be removably attached to the lid 212 (e.g., using semi-permanent adhesive). Removable attachment can allow a user to remove or replace the light block.

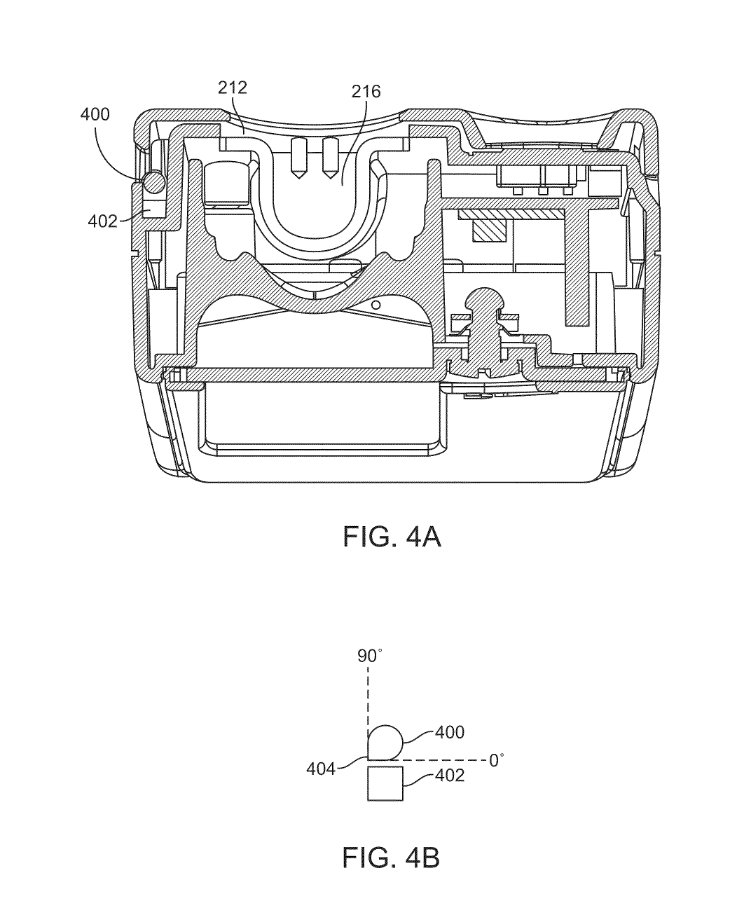

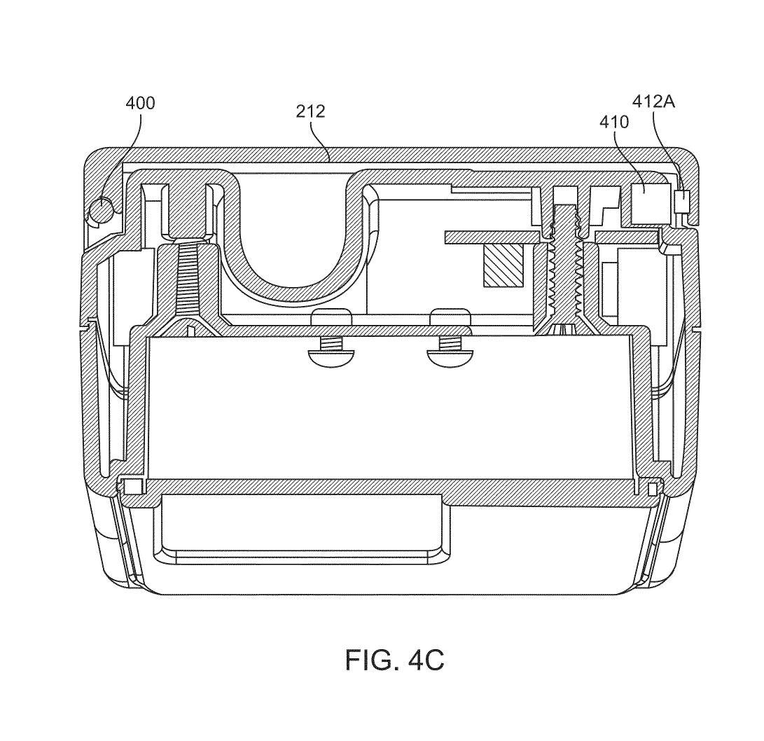

As shown in FIGS. 2B and 3B, the lid hinge can be flush with the overall shape of the device. There is no hinge barrel protruding from the unit 200. This can help reduce device profile and increase comfort for the user when the unit 200 is positioned on or against the body. FIGS. 4A-B illustrate an embodiment of a magnetic hinge.

FIG. 4A shows a cross sectional view of the unit 200, including a hinge pin 400 and magnet 402 positioned on one side of the unit 200. The hinge pin 400 and magnet 402 can operate as a hinge. Instead of the pin 400 being fixed in place, the magnet 402 can hold the pin 400 in place. The magnetic holding of the pin 400 allows breakaway with no or minimal damage to components of the unit 200. Additionally, the magnetic hinge can allow the lid to be easily replaced or customized. Other hinge configurations are also possible. For example, a traditional hinge pin can be used.

FIG. 4B illustrates a close up view of just a pin 400 and magnet 402. A flat portion 404 on the pin 400 can encourage the lid 212 to stay in the closed position. The flat portion 404 can also provide tactile feedback to a user as the user moves the lid through various positions. Additional flat portions can provide tactile feedback to encourage stopping at one or more lid positions.

The pin 400 can comprise stainless steel (e.g., 416 SS). This material can be both corrosion resistant and magnetic. Other materials are also possible. For example, the pin 400 can comprise plated mild steel. The pin can be hollow or stamped.

In some embodiments, the pin 400 can be attached to the lid 212. Possible attachment methods can include adhesives, press fit, snap fit, or heat staking.

FIG. 4C illustrates a cross-sectional view of the unit 200 showing the lid clasp. A housing magnet 410 can be positioned to interact with a lid magnet 412A positioned at the front of the lid, away from the hinge portion of the lid. The interaction between the magnets can hold the lid in a closed position. In some embodiments, the lid 212 and/or the housing 202 can include more than one magnet to hold the lid 212 in a closed position.

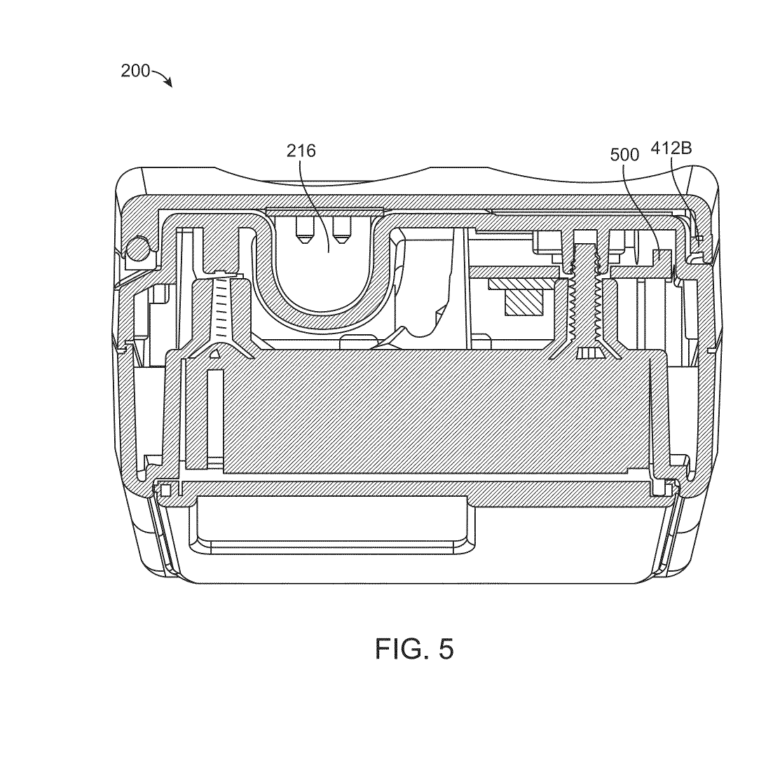

FIG. 5 illustrates a cross sectional view of the unit showing an embodiment of a lid closed sensor. A lid magnet 412B can be configured to interact with a reed switch 500 mounted on the circuit board (described in more detail below). The reed switch 500 can provide feedback indicating a closed position to an internal monitoring system. The unit 200 can be configured to be disabled and not provide UV disinfection energy until the lid is closed. Other lid close sensors are also possible. For example, hall effect, optical or a microswitch can be used.

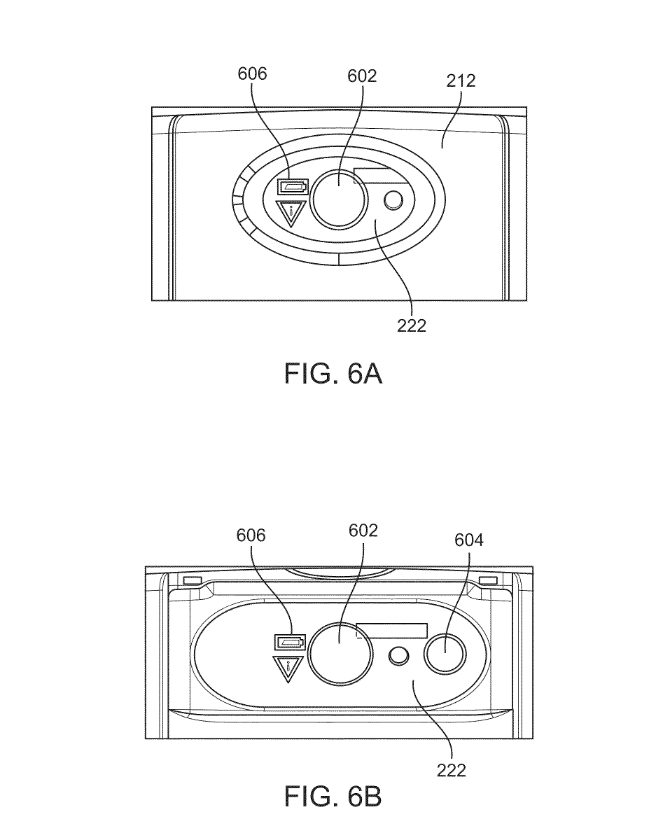

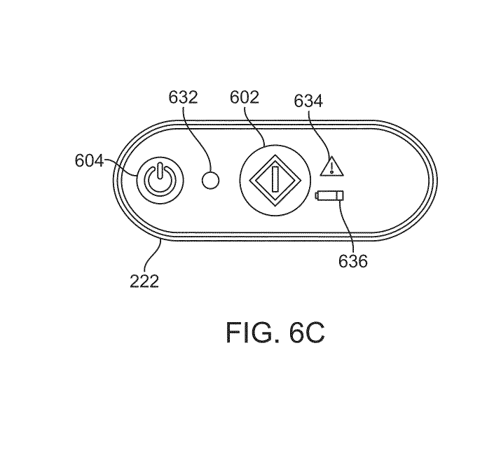

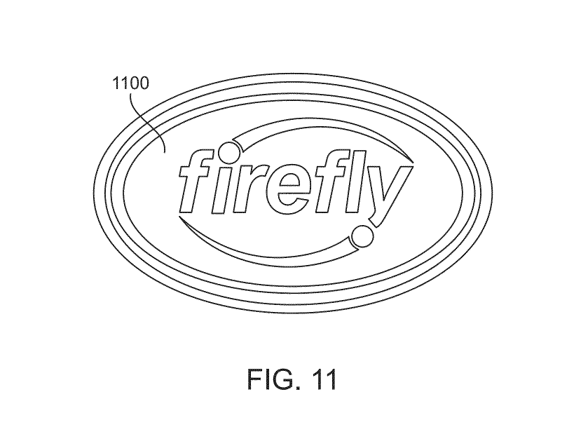

FIGS. 6A and 6B illustrate an embodiment of the control panel 222 positioned on the upper housing 201 beneath lid opening 220. FIG. 6A shows the control panel 222 with the lid 212 down, while FIG. 6B shows the control panel 222 with the lid up. The control panel includes an activation button 602 and a power button 604. As shown in FIG. 6A, the power button 604 is not accessible with the lid 212 closed. Positioning the power button 604 beneath the lid can prevent access to the power button in the closed position when the device may be in storage or transport. This feature can help extend the lifespan of the lamps and the batteries. The control panel 222 can comprise a flex panel overlay. This overlay can provide protection from water ingress and provide UV blocking functionality. The symbols 606 on the control panel can provide feedback of the device state. The control panel 222 can be backlit by the UV light source of the device. This UV backlighting can allow a user to verify UV output. The device can also have a logo 1100 on the lid 212 that is backlit by the UV light source (FIG. 11).

FIG. 6C shows another embodiment of the control panel 222 by itself. The panel comprises a power button 604, a power on lamp indicator 632, an activation button 602, an error indicator 634, and a low battery indicator 636.

The control panel can be about 0.6-1.0″ wide. For example, the control panel can be about 0.8″ wide. The control panel 222 can be about 2.0-2.4″ long. For example, the control panel can be about 2.0-2.4″ wide. The panel 222 can have rounded ends, creating an ovular shape. Other shapes (e.g., rectangular, square) are also possible.

The lid 212 can comprise a UV opaque material such as polycarbonate, ABS or polyethylene. The lid 212 can act as a light block to prevent UV light from travelling outside the unit 200.

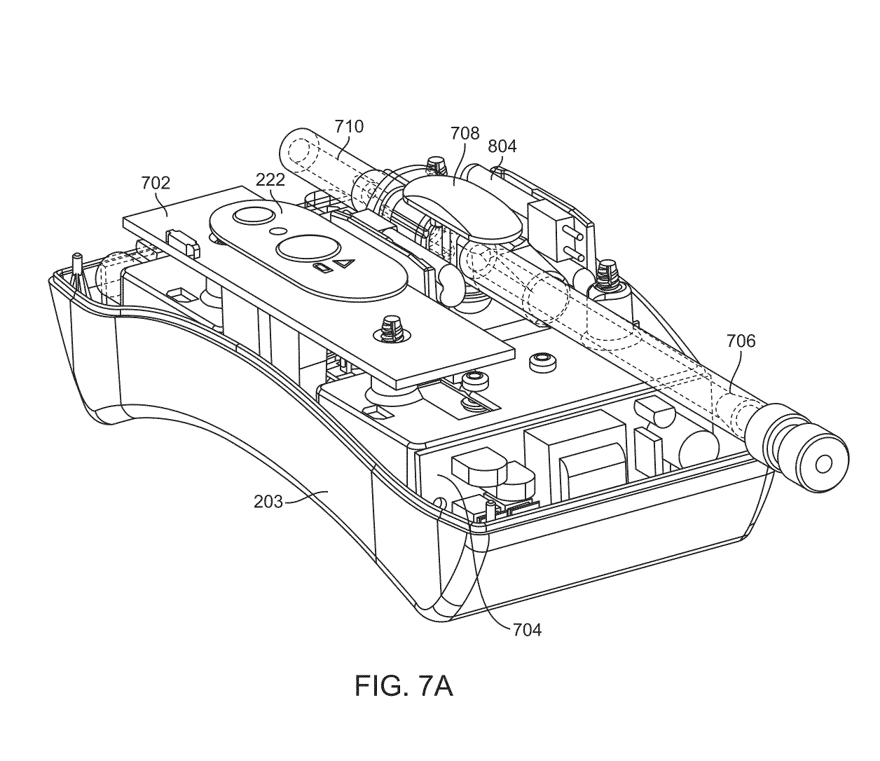

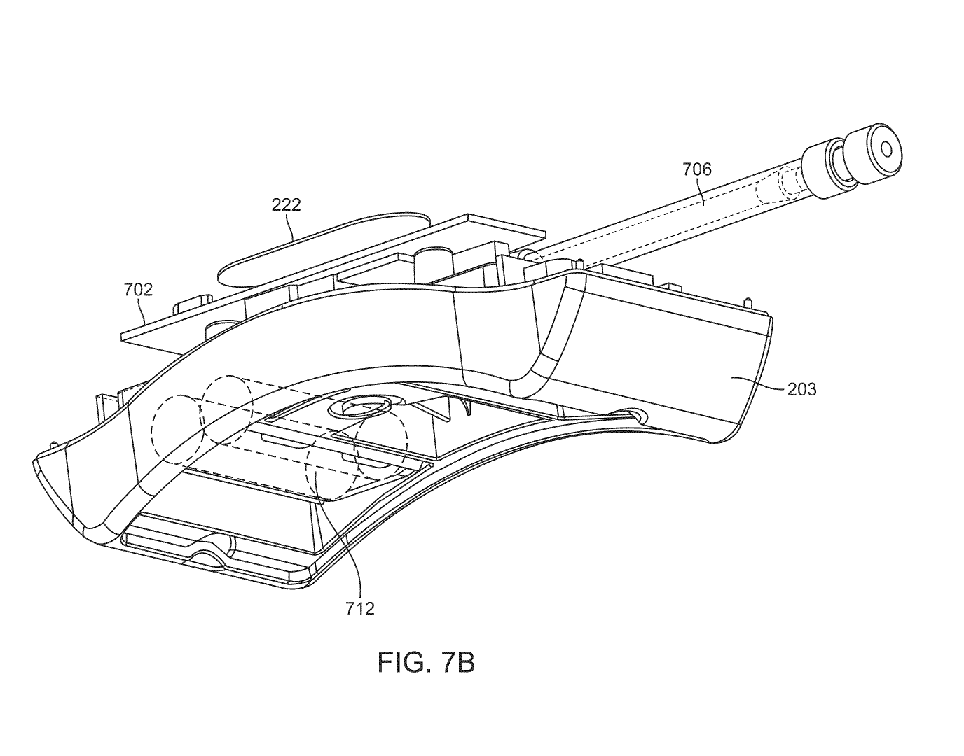

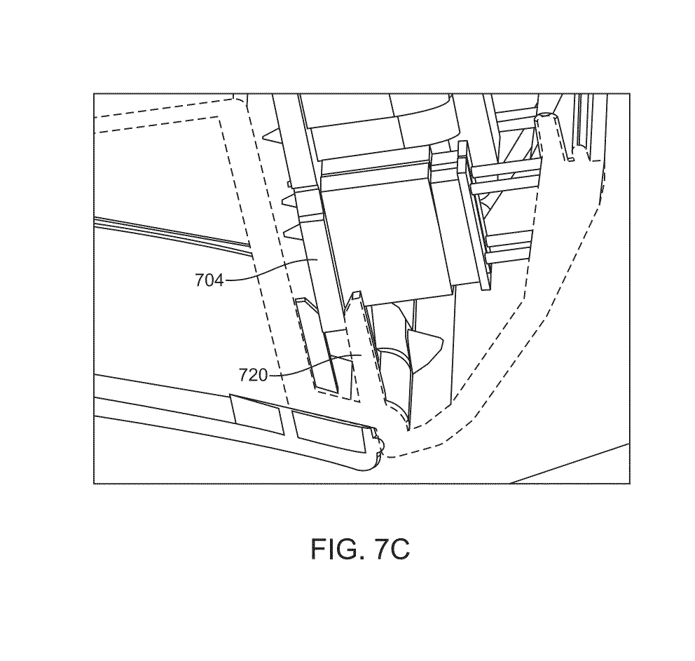

FIGS. 7A and 7B show the unit 200 with the top housing 201 removed, leaving only the bottom housing 203, allowing visualization of internal components. FIG. 7A is a top perspective view showing the control panel 222 separated from the top housing 201. FIG. 7A also shows partial view of the lamps 804, a circuit board 702, and one of two inverter boards 704. A catheter 706 comprising transfer valve 708, described in the '433 Application is shown positioned within the unit 200. The transfer valve 708 is shown connected to a solution set catheter 710.

FIG. 7B is a bottom perspective view of the unit 200 with the battery door 904 and top housing 201 removed. FIG. 7B shows where the two of the four batteries 712 are positioned. FIG. 7B depicts 2 AA batteries, but other batteries and amounts of batteries are also possible. In some embodiments, the unit 200 may be plugged into a wall outlet.

The inverters 704 of the unit 200 can be held in place by ribs 720, as shown in FIG. 7C. FIG. 7C, a cross-sectional view of the end of unit 200, depicts ribs 720 holding inverter 704 in place. Ribs can allow internal components to be held in place without fastening features. The ribs 720 can be thin and flexible to allow components to slide in position and secure them in place.

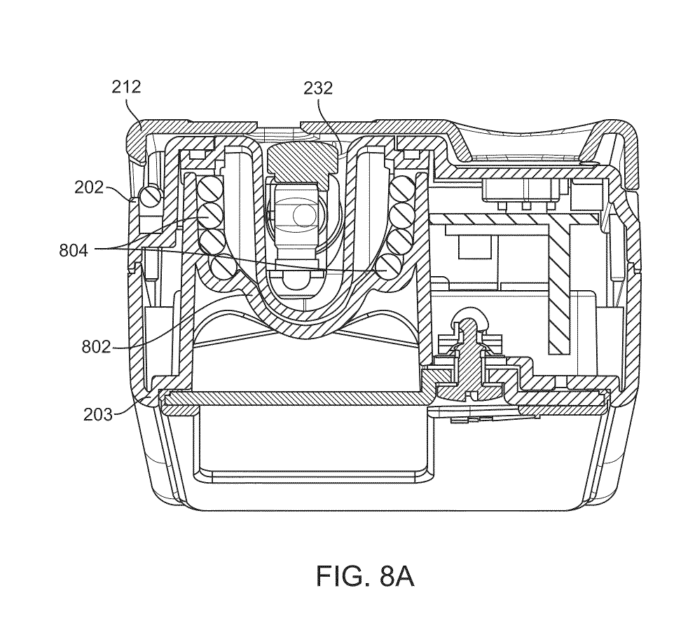

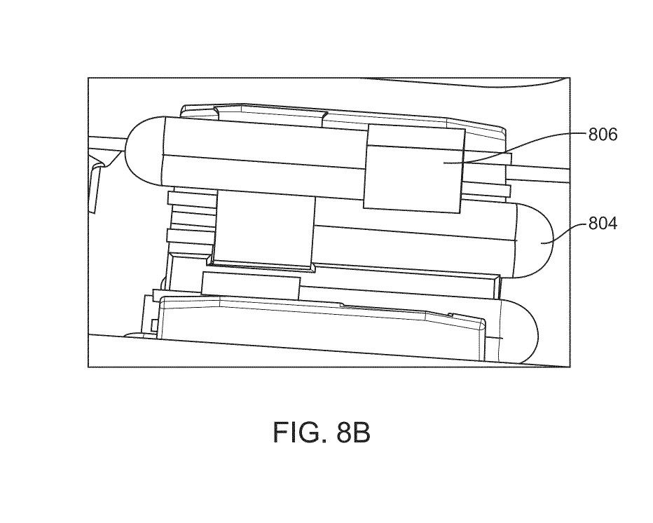

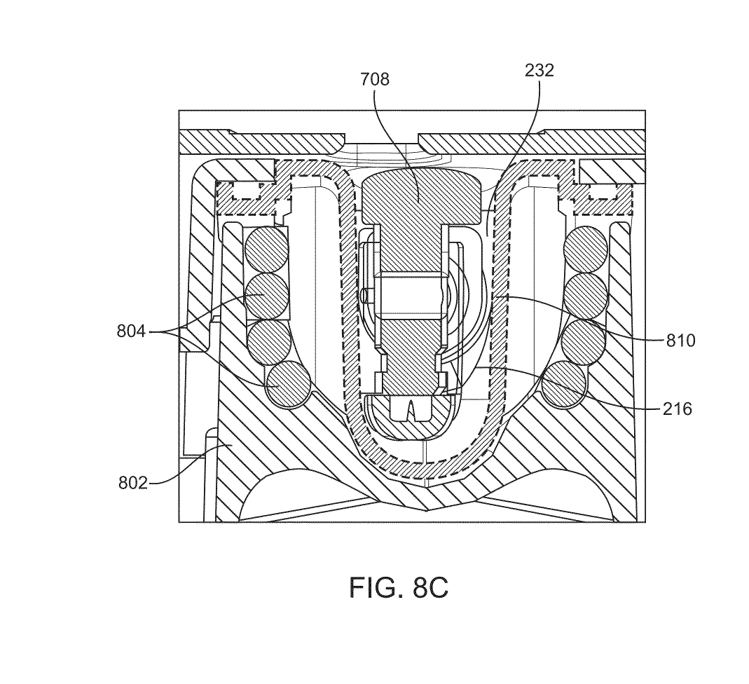

FIG. 8A illustrates a cross sectional view of the unit 200 showing the lamp support 802 integrated as part of the lower housing 203. The lamp support 802 can hold the lamps 804 in place between the lower housing 203 and the exposure chamber 232. The lamp support 802 can also provide a clamping force for the exposure chamber between a bottom and top portion of the housing 202.

FIG. 8B illustrates a perspective view of the lamps 804. As shown, the lamps 804 can be offset with alternating orientation. Such a configuration can enhance UV fluence. Other configurations are also possible. For example, the lamps can be oriented substantially vertically instead of horizontally, or the multiple lamps can be replaced by one or more serpentine lamps. FIG. 8B also depicts lamp electrodes 806.

FIG. 8C illustrates a close up view of the exposure chamber 232 surrounded by the lamps 804. The lamps 804 are positioned on either side of the exposure chamber. FIGS. 8A-8C show 2 lamps positioned on either side of the exposure chamber 216. Fewer or more lamps are also possible. For example, in some embodiments, 1, 3, 4, 5, or 6 lamps can be used. Lamps with varying configurations and shapes are also possible. For example, curved or helical lamps can be used.

The lamps can comprise readily available mercury vapor cold cathode fluorescent lamps (CCFL), or UV emitting LEDs, such as those available from Sensor Electronic Technology Incorporated, or xenon strobe lamps (xenon lamps produce a burst of UV light at start-up).

A light transmitting surface 810 is shown positioned between the exposure chamber 232 and the lamps 804. The light transmitting surface 810 defining the exposure chamber 232 can comprise a UVC transparent material (e.g., TPX, COC, quartz). In some embodiments, the light transmitting surface 810 is integrally formed as part of the central portion of the tubing trough 216 such as via injection molding. Alternatively, light transmitting surface 810 can be a separately applied UV transparent window. In either case, the light transmitting surface can be about 1 mm thick. The planar shape of the surface 810 adjacent to the lamps 804 can help to allow maximum light transparency to the exposure chamber 232, which can help minimize reflection and refraction.

The lamps can be positioned about 0-1 cm away from the light transmitting surface. Having the lamps 804 closer to surface 810 can help maximize the flux of the UV light output, and help minimize divergence.

FIG. 8C illustrates a transfer valve 708, described in the '433 Application, positioned in the exposure chamber 232. As described in the '433 Application, the transfer valve 708 can be configured such that any fluid in the valve to be disinfected is positioned on an exterior portion of the valve so it can be exposed to UV light.

As shown in FIGS. 7A and B, the unit 200 can comprise a processor configured to provide memory and control functionality to the unit 200. The processor can be part of a circuit board 702 positioned within the unit 200. In some embodiments, processing can take place outside of the device. In some embodiments, the device can be configured to communicate with an external processor.

The processor can be configured to control the duty cycle of the lamps and help prevent overheating of the lamps. For example the processor can add 4 “units” of lamp on-time (per second of real time) to a register while simultaneously and continuously subtracting 1 “unit” of off-time (per second of real time) from the same register. If the total “units” in the register exceeds a pre-set value, power to the lamps is terminated until the total “units” return to below the pre-set value. In this example the duty cycle would be approximately 20%. Other duty cycles are possible. For example, duty cycles can be governed by the following relationship:

$$Duty Cycle = \frac{1}{on \text{-} time unit modifier + 1}$$

Examples of on-time unit modifiers: duty cycles include 1:50%, 3:25%, 4:20%, and 9:10%. Calculating duty cycle in this manner can allow the duty cycle to be controlled without requiring extensive processing power. Alternatively, the duty cycle can be controlled using power and time measurements.

The processor can comprise a memory unit which can be configured to record data regarding the unit 200. For example, the processor can be configured to record data relating to disinfection time, disinfection duration, disinfection power, duty cycle, etc.

The processor can comprise overtemperature protection functionality. This can be done using sensors (e.g., heat sensor) or logic. For example, the processor can be configured to prevent use of the unit 200 when the ambient temperature is undesirably high (e.g., above a predetermined limit).

The processor can be configured to provide status and error signals to the user. For example, the processor can be configured to receive information from the lid sensor (monitoring reed switch 500). If the lid sensor sends a signal indicating that the lid is open, the processor can be configured to send an error signal to the user. The error signal can be shown on the control panel 222. The processor can also be configured to detect when a lamp goes out. For example, the processor can be configured to monitor inverter current to detect a lamp not functioning.

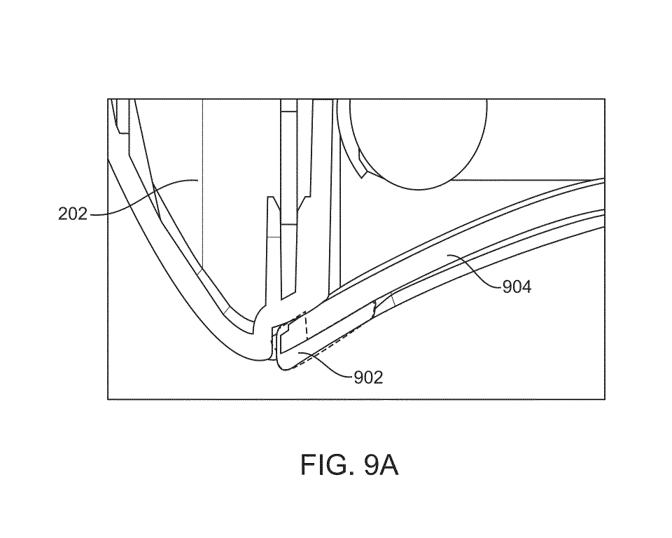

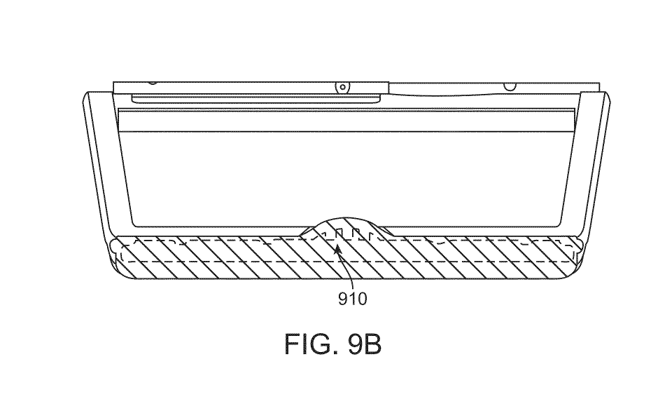

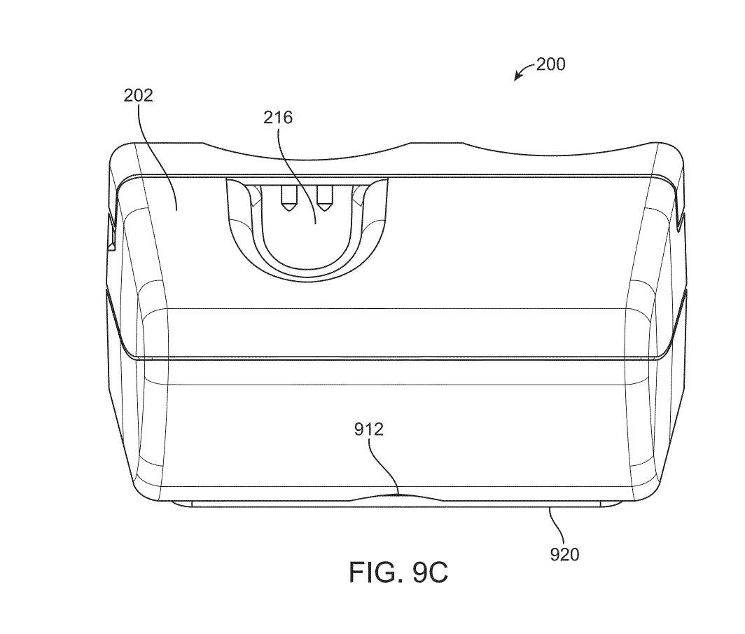

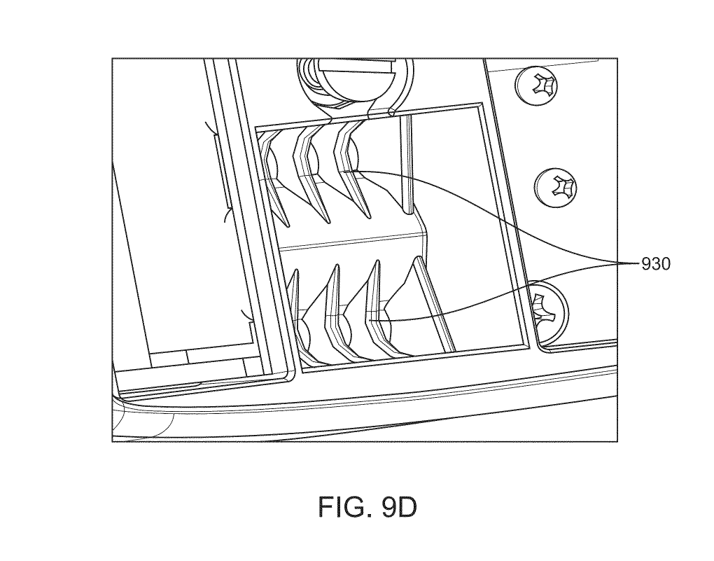

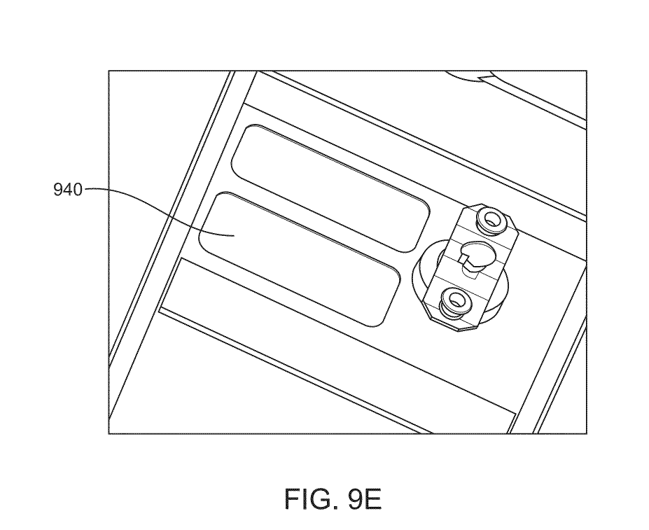

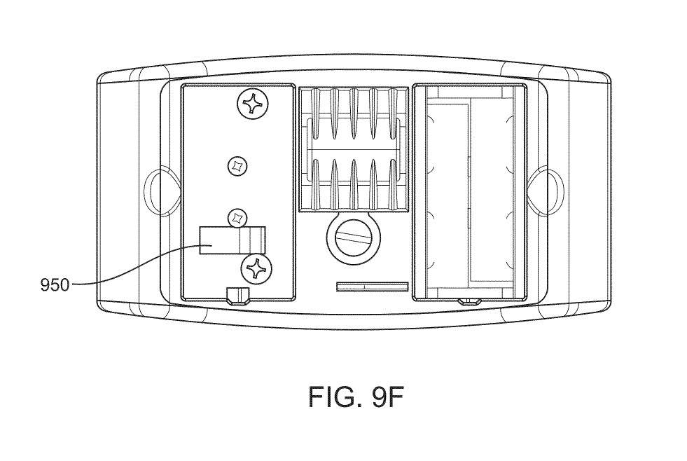

FIG. 9A illustrates a cross-sectional view of a bottom corner of housing 200. This view illustrates in the interaction between the bottom housing 203 and the battery door 904. Within this view can be seen structure 902 on the battery door 904 that can be a soft elastomeric feature. This can help add comfort to the user during use of the unit 200. The feature 902 can also provide a sealing surface along with the bottom housing 203, preventing escape of light from the unit 200 and ingress of light and contaminants into the unit 200.

FIG. 9B illustrates a rib structure 910 in battery door 904 that can provide a feature to aid in removing the battery door from the housing bottom 203. FIG. 9C also shows finger reliefs 912 on the unit 200, allowing access to the battery door 904. The rib structure 910 can prevent sinks when the soft elastomeric material is overmolded onto the battery door.

FIG. 9C illustrates an anti skid feature 920 on the unit 200. The feature 920 can comprise a soft elastomeric material configured to engage flat surfaces to prevent sliding. The feature 920 can also provide grip to the unit when placed on the thigh or lap of a user.

FIG. 9D illustrates ribs 930 on the underside 204 of the bottom housing 203 that can hold spare caps in place. The caps can be used by a catheter/valve used with the disinfection unit 200, as described in the '433 Application. Pads on the battery door can also help to hold the caps in place.

FIG. 9E illustrates pads 940 positioned on the underside of the battery door 904. The pads 940 can hold the caps in place without rattling.

FIG. 9F illustrates an access hole 950 underneath the battery case within bottom housing 203. The access hole 950 can allow access for data and screws for disassembly. The positioning of the access hole 950 can be unobvious to the user and help prevent tampering.

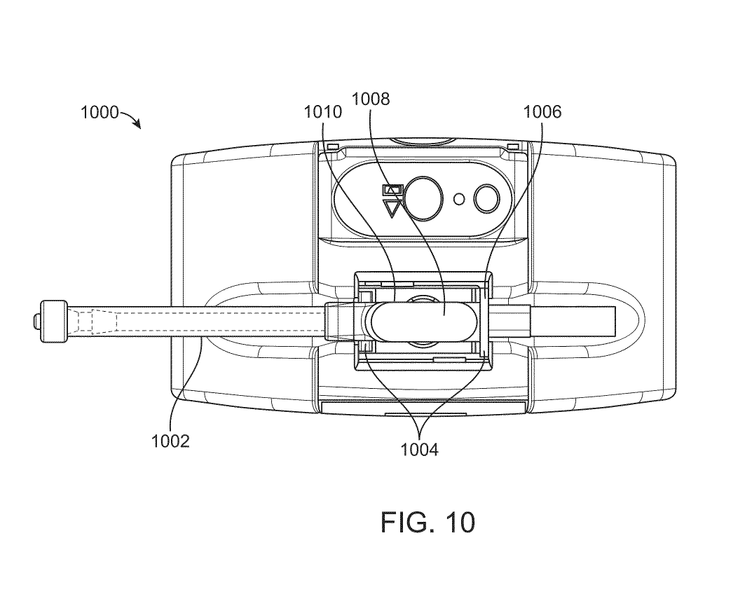

FIG. 10 illustrates another embodiment of a disinfection device 1000. FIG. 10 illustrates a catheter connection 1002 positioned within the device 1000. Recesses 1004 within the tubing trough are configured to receive a feature, e.g., a shoulder 1006, of the catheter connection, helping to position and maintain the valve 1008 of the catheter connection within the exposure chamber 1010.

FIG. 11 illustrates an embodiment of a graphic overlay 1100 which can be used with embodiments of a disinfection device. The overlay 1100 can be configured to be backlit by the UV source, thereby indicating activation to a user. Firefly is used as an exemplary logo. Other embodiments are contemplated.

It will be appreciated that while the disinfection device has been described in connection with peritoneal dialysis, the transfer catheter and/or valve can be used in numerous other applications, medical or otherwise.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. An ultraviolet (UV) disinfection unit for a catheter line connection, comprising
a housing comprising a concavely curved bottom surface;
a lid shaped to mate with a top open part of the housing, the lid connected to the housing by a hinge;
a UV source positioned within the unit;
a tubing trough positioned beneath the lid and configured to receive a catheter connector connecting two catheters; and
an exposure chamber positioned along the tubing trough, the exposure chamber at least partially exposed to the UV source.

2. The disinfection unit of claim 1, further comprising a first recess positioned along the tubing trough on a first side of the exposure chamber and a second recess positioned along the tubing trough on a second side of the exposure chamber, the first and second recesses shaped to receive a protrusion on the catheter line connection and stabilize the catheter line connection within the tubing trough.

3. The disinfection unit of claim 1, wherein the UV source comprises 4 lamps.

4. The disinfection unit of claim 1, wherein the unit comprises a lid close sensor.

5. The disinfection unit of claim 1, wherein the hinge comprises a magnetic hinge.

6. The disinfection unit of claim 1, wherein the exposure chamber comprises a volume of about 15 cc.

7. The disinfection unit of claim 1, comprising a volume of about 5.5 in³.

8. The disinfection unit of claim 1, wherein the lid comprises a graphic overlay configured to be backlit by the UV source.

9. The disinfection unit of claim 1, wherein the lid is configured to block access to a power control when the lid is in a closed position.

10. The disinfection unit of claim 1, wherein the hinge comprises a pin comprising a flat portion configured to encourage the lid to stay in a closed position.

11. A UV disinfection unit for a catheter line connection, comprising
a housing;
a lid shaped to mate with a top open part of the housing, the lid connected to the housing by a hinge;
a UV source positioned within the unit;
a tubing trough positioned beneath the lid and configured to receive a catheter line connection;
an exposure chamber positioned along the tubing trough, the exposure chamber at least partially exposed to the UV source;
a protrusion along the catheter line connection;
a recess positioned along the tubing trough within the exposure chamber, the recess shaped to receive the protrusion and stabilize the catheter line connection within the tubing trough.

12. The disinfection unit of claim 11, comprising a first recess positioned along the tubing trough on a first side of the exposure chamber and a second recess positioned along the tubing trough on a second side of the exposure chamber, the first and second recesses shaped to receive the protrusion and stabilize the catheter line connection within the tubing trough.

13. The disinfection unit of claim 11, comprising a concavely curved bottom surface.

14. The disinfection unit of claim 11, wherein the exposure chamber is configured to receive a transfer valve comprising external UV-transparent channels.

15. A UV disinfection unit for a catheter line connection, comprising
a housing;
a lid shaped to mate with a top open part of the housing, the lid connected to the housing by a hinge;
a UV source positioned within the unit;
a tubing trough positioned beneath the lid and configured to receive a catheter line connection;
an exposure chamber positioned along the tubing trough, the exposure chamber at least partially exposed to the UV source;
a protrusion along the catheter line connection;
a first recess positioned along the tubing trough on a first side of the exposure chamber, a second recess positioned along the tubing trough on a second side of the exposure chamber, the first and second recesses shaped to receive the protrusion and stabilize the catheter line connection within the tubing trough.

16. The disinfection unit of claim 15, wherein the UV source comprises 4 lamps.

17. The disinfection unit of claim 15, wherein the unit comprises a lid closed sensor.

18. The disinfection unit of claim 15, wherein the hinge comprises a magnetic hinge.

19. The disinfection unit of claim 15, wherein the exposure chamber comprises a volume of about 15 cc.

20. The disinfection unit of claim 15, comprising a volume of about 5.5 in³.

21. The disinfection unit of claim 15, wherein the lid comprises a graphic overlay configured to be backlit by the UV source.

22. The disinfection unit of claim 15, wherein the lid is configured to block access to a power control when the lid is in a closed position.

23. The disinfection unit of claim 15, wherein the housing comprises a concavely curved bottom surface.

24. A method for disinfecting a catheter line connection, comprising
placing a UV disinfection unit on a thigh of the user such that the concavely curved bottom surface of the unit is positioned on the user's thigh;
positioning a catheter line connection within a tubing trough of the unit such that the portion to be disinfected is placed within an exposure chamber comprising a portion of the tubing trough deeper than other portions; and
exposing the exposure chamber to UV light.

25. The method of claim 24, further comprising opening a lid of the disinfection unit to access a power control.

26. The method of claim 24, further comprising preventing overheating of a UV lamp positioned within the unit.

27. The method of claim 26, wherein preventing overheating comprises adding 4 units of lamp on-time per second of real time to a register while simultaneously and continuously subtracting 1 unit of lamp off-time per second of real time.

28. The method of claim 27, further comprising terminating power to the lamps if the total units in the register exceeds a pre-set value.
